(19) United States
(12) Patent Application Publication
(10) Pub. No.: US 2005/0129610 A1
(43) Pub. Date: Jun. 16, 2005

(54) Imaging agents and methods of imaging alpha7-nicotinic cholinergic receptor

(75) Inventors: Martin G. Pomper, Baltimore, MD (US); John L. Musachio, Baltimore, MD (US); Hong Fan, Lutherville Timonium, MD (US); Robert F. Dannals, Sparks, MD (US); Catherine Foss, Baltimore, MD (US); Eifion Phillips, Wilmington, DE (US); John Gordon, Wilmington, DE (US); Dennis McCarthy, Wilmington, DE (US); Richard Keith, Wilmington, DE (US); Mark Smith, Wilmington, DE (US); Richard Heys, Wilmington, DE (US); Peter N. Dorff, Wilmington, DE (US)

Correspondence Address: EDWARDS & ANGELL, LLP, P.O. BOX 55874, BOSTON, MA, 02205, US

(73) Assignee: Johns Hopkins University

(21) Appl. No.: 10/877,813

(22) Filed: Jun. 24, 2004

Related U.S. Application Data
(60) Provisional application No. 60/482,108, filed on Jun. 24, 2003.

(51) Int. Cl. A61K 51/00; A61K 31/4747; C07D 491/14; C07F 5/00

(52) U.S. Cl. 424/1.11; 514/278; 534/11; 546/18

(57) ABSTRACT

The present invention relates to radiolabelled compounds particularly 1-azabicyclo[2.2.2]octane compounds (i.e., quinuclidine compounds) which are labeled with one or more radioisotopes and which are suitable for imaging or therapeutic treatment of tissues, organs, or tumors which express the a7-nicotinic cholinergic receptor. In another embodiment, the invention relates to methods of imaging tissues, organs, or tumors using radiolabeled compounds of the invention, particularly tissues, organs, or tumors which express a7-nicotinic cholinergic receptor to which the compounds of the invention have an affinity.

This application claims the benefit of U.S. Provisional Application Ser. No. 60/482,108 filed Jun. 24, 2003, the teachings of which are incorporated herein by reference.

### BACKGROUND OF THE INVENTION

1. Field of the Invention

The present invention provides novel radiolabeled quinuclidine compounds and more particularly spiro(quinuclidine-dihydrofuran) and N-quinuclidinyl-benzamide compounds capable of binding with high selectivity and/or high affinity to nicotinic cholinergic receptors and more preferably high selectivity for the α7 nicotinic cholinergic receptor. This invention also provides pharmaceutical compositions comprising such radiolabeled quinuclidine compounds. Additionally this invention provides imaging methods for localizing nicotinic cholinergic receptors in tissues or cells using radiolabeled quinuclidine compounds of the invention. The invention further provides treatment methods comprising administration of a high energy radiolabelled quinuclidine compounds to a patient, particularly patients suffering from diseases or disorders associated with over-expression or under-expression of one or more nicotinic cholinergic receptors such as schizophrenia, Alzheimer's disease, and lung cancer.

2. Background

The α7 nicotinic cholinergic receptor (α7-nAChR) is a cationic, ligand-gated calcium channel with five identical ligand-binding subunits. Along with the α4β2-nAChR subtype, the α7-nAChR represents the most abundant nAChR in the brain. Presynaptically, α7-nAChRs modulate transmitter release, postsynaptically they are excitatory and generate depolarizing currents and perisynaptically they provide a neuromodulatory function (Berg, et al., J Neurobiol 2002;53(4):512-23.). Additional pharmacology indicates that α7-nAChRs influence neurite outgrowth, activate second messenger systems and facilitating memory, i.e., long-term potentiation (Pugh, et al., J Neurosci 1994;14(2):889-96).

The regulation of calcium-related events by α7-nAChRs underlies the ability for this nicotinic receptor subtype to modulate glutamatergic and nitrergic transmission (Si, et al., Circ, Res.2002;91(1):62-9). The α7-nAChR mediates increases in tyrosine hydroxylase expression upon activation (Serova, et al., J Pharmacol Exp Ther 2002;303(3):896-903), conferring neuroprotection via the Src, Akt, Bcl-2/Bcl-x pathway (Kihara, et al., J Biol Chem 2001;276(17):13541-6) and facilitates amyloid deposition within neurons (Nagele, et al., Neuroscience 2002;110(2):199-211).

In the central nervous system, α7-nAChRs are involved in sensory gating, memory and neuronal plasticity. α7-nAChRs have also been implicated in a wide variety of pathological states including Alzheimer disease (Kem, et al., Behav Brain Res 2000;113(1-2):169-81), schizophrenia (Freedman, et al., Curr. Psychiatry Rep 2003;5(2):155-61), aging (Perry, Alcohol 2001; 24(2):63-8), head trauma (Verbois, et al., Neuropharmacology, 2003; 44(2):224-33), inflammation (Wang, et al., Nature 2003; 421(6921):384-8), nicotine addiction (Buisson, et al., Trends Pharmacol Sci, 2002; 23(3):130-6), and lung cancer (Song, et al., Cancer Res., 2003; 63(1):214-21). Notably, while the number of α4β2-nAChRs is decreased in Alzheimer disease, the number α7-nAChRs remains largely intact and therefore available for binding new therapeutic agents.

¹²⁵I-α-bungarotoxin (¹²⁵I-α-BTX) and ³H-methyllycaconitine (³H-MLA) are commonly used in vitro and ex vivo as radio-imaging agents to characterize α7-nAChR, but neither of these imaging agents exhibits complete binding selectivity for the α7-nAChR receptor (Whiteaker, et al., Eur J Neurosci 1999;11(8):2689-96; and Marinou, et al., Biochem. J. 2003;372(Pt 2):543-54). ³H-MLA labels 21% more specific sites than does ¹²⁵I-α-BTX, which displays slow binding kinetics due to its large size. These agents have been used to localize the α7-nAChR in the pons, hippocampus and colliculi in the mouse brain.

Specific ligands used to study α7-nAChRs range in size from that of the protein, α-BTX, to small molecules, e.g., AR-R17779 which has a molecular weight of less than 200 g/mol.

Various structurally divergent compounds have been shown to bind to α7-nAChRs selectively. These selective α7-nAChR ligands include certain oxystilbenes (Gotti, et al., Behav Brain Res 2000;113(1-2):183-92), anabaseines (Uteshev, et al., J Neurophysiol 2003;89(4):1797-806), and quinuclidines (Mullen, et al., J Med Chem 2000;43(22):4045-50). Not withstanding the structural diversity of identified a 7-nAChR ligands, structure-activity relationships pursued with the quinuclidine series indicated a low tolerance for structural modification of putative ligands.

International patent application, PCT/SE98/01364 teaches a series of substituted quinuclidine and structurally related azabicyclo compounds, the use of the recited compounds for nicotinic acetylcholine receptors, and treatment of certain neurological disorders. However, the cited patent does not teach or suggest radiolabelled compounds or methods of imaging using such radiolabelled derivatives.

While a variety of compounds have been synthesized as α7-nAChR ligands, only two have been synthesized in radiolabeled form for imaging, i.e., [¹¹C]AR-R255082 and 3-({2,4-dimethyl-5-[¹²³I]iodo}benzylidene)anabaseine, with neither of those demonstrating any regionally selective binding in vivo (Gotti, et al., Behav Brain Res 2000;113(1-2):183-92; and Broad, et al., Eur J Pharmacol 2002;452(2):137-44).

It would be desirable to have a family of compounds, including radiolabeled compounds, having high affinity for α-7 nicotinic cholinergic receptor, which can be readily prepared.

### SUMMARY OF THE INVENTION

The invention provides novel radiolabelled quinuclidine compounds of Formula I, and pharmaceutical compositions comprising compounds of Formula I and at least one pharmaceutically acceptable carrier or excipient. Preferred radiolabelled compounds of the invention exhibit high affinity for nicotinic cholinergic receptors, and more particularly, the α7 subtype nicotinic cholinergic receptors.

In a preferred aspect, the invention provides radiolabelled quinuclidine compounds according to Formula I 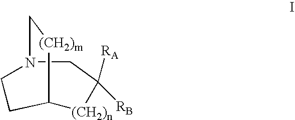
wherein m and n are independently selected from 0 or 1; R_{A} is NR₅R₆; R_{B} is hydrogen, C₁alkyl, C₂₋₆alkenyl, C₂₋₆alkynyl, or (C₃₋₈cycloalkyl)C₀₋₆alkyl; or R_{A} and R_{B}, taken in combination, form a substituted heterocycle or a substituted benzoheterocycle; and R₅ and R₆ are independently selected from hydrogen, C₁alkyl, C₂₋₆alkenyl, C₂₋₆alkynyl, optionally substituted phenyl, optionally substituted benzyl, optionally substituted benzoyl, optionally substituted 5- to 7-membered heteroaryl, wherein the compound of Formula I comprises at least one radioisotope; or a pharmaceutically acceptable salt thereof.

The present invention provides radiolabelled quinuclidine compounds of Formula I and subformula thereof which are ligands for nicotinic cholinergic receptors and are suitable for use in imaging or radiotherapeutic applications. More particularly, the invention provides imaging agents comprising a radiolabelled quinuclidine of the invention which has one or more radioisotopes of carbon, fluorine, iodine, or technetium which is capable of binding to α7 subtype of the nicotinic cholinergic receptors. Certain preferred radiolabeled quinuclidine compounds of the invention are suitable for use in localizing α7 subtype of the nicotinic cholinergic receptors in vivo under a variety of conditions wherein the radiation emitted by the radioisotope of the radiolabelled quinuclidine is utilized to form the image. In preferred embodiments, radiolabelled quinuclidine compounds of the invention comprise one or more radioisotopes capable of emitting positron radiation and are suitable for use in positron emission tomography (PET) or single photon emission computed tomography (SPECT).

One class of radiolabelled quinuclidine compounds provided by the present invention includes those compounds prepared by chemical modification of a non-radiolabeled compound disclosed in WO 99/03859. In certain other preferred aspects, radiolabelled quinuclidine compounds of the invention comprise a 3-amino or 3-benzamido substituted quinuclidine which is modified to incorporate one or more radioactive isotopes of carbon, fluorine, iodine, or technetium.

According to yet another aspect, the present invention provides pharmaceutical compositions comprising radiolabeled compounds of Formula I or the pharmaceutically acceptable salts or solvates thereof, which compositions are useful for the imaging of the above-recited enzymes or receptors, tissues expressing said enzymes, tumors or angiogenesis. The invention further provides methods of imaging patients suffering from any of the above-recited disorders or disorders with an effective amount of a compound or composition of the invention.

Additionally this invention relates to the use of the compounds of the invention (particularly labeled compounds of this invention emitting high energy radiation) as therapeutic agents for the treatment of diseases and disorders associated with elevated expression of enzymes or receptors for which the radiolabelled quinuclidine compounds of the invention have high binding affinity, e.g., disorders or diseases associated with elevated or reduced nicotinic cholinergic receptor expression. Typical disease and disorders include cancer, tumors, stroke, collagen vascular disease, vascular malformations, normal tissue growth, and the like.

Preferred radiolabelled quinuclidine_compounds of the invention exhibit good binding activity and/or affinity for nicotinic cholinergic receptor. Particularly preferred radiolabelled quinuclidine compounds of the invention are α7-subtype nicotinic cholinergic receptor inhibitors having a Kᵢ of about 1 micromolar or less, still more preferably a Kᵢ of about 100 nanomolar, 50 nanomolar or less or even more preferably a Kᵢ of about 10 nanomolar or less.

Other aspects of the invention are disclosed infra.

### BRIEF DESCRIPTION OF THE DRAWINGS

FIG. 1 is a graph of brain kinetics of compound three, e.g., a plot of uptake of compound 3 in the cerebellum (CB), hippocampus (HIPP), and cortex (CTX) at a series of time intervals post injection;

FIG. 2 is a graph of binding specificity for compound 3 in the cerebellum (CB), hippocampus (HIPP), striatum (STR), and cortex (CTX) in the presence and absence of a nicotine blockade;

FIG. 3 is a graph of brain kinetics of compound three, e.g., a plot of uptake of compound 4 in the cerebellum (CB), hippocampus (HIPP), and cortex (CTX) at a series of time intervals post injection; and

FIG. 4 is a graph of binding specificity for compound 4 in the cerebellum (CB), hippocampus (HIPP), striatum (STR), and cortex (CTX) in the presence and absence of a series of nicotine concentration blockades.

### DETAILED DESCRIPTION OF THE INVENTION

In addition to compounds of Formula I, described above, the invention is further directed to compounds and pharmaceutically acceptable salts of Formula I (shown above) wherein the compounds provided by the invention are compounds and salts of Formula II.

In certain aspects, preferred compounds of Formula I include those compounds having a structure according to Formula II 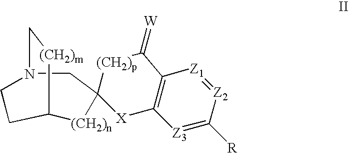
wherein
m, n, and p are independently selected from 0 or 1;
W is O, F₂ or H₂;
X is O or S;
Z₁ is N or CR₁;
Z₂ is N or CR₂;
Z₃ is N or CR₃, wherein one or two of Z₁, Z₂, and Z₃ is nitrogen;
R is halogen, C₁₋₆haloalkyl, C₁₋₆alkoxy, C₁₋₆haloalkoxy, C₁₋₆alkylthio, formyl, carboxylate, —NR₅R₆, substituted aryl, or substituted heteroaryl;
R₁, R₂, and R₃ are independently selected at each occurrence from the group consisting of hydrogen, halogen hydroxy, amino, cyano, nitro, C₁₋₆alkyl, C₂₋₆alkenyl, C₂₋₆alkynyl, C₁₋₆alkoxy, mono- or di-C₁₋₆alkylamino, optionally substituted aryl or optionally substituted heteroaryl; and
R₅ and R₆ are independently selected from hydrogen, C₁₋₆alkyl, C₂₋₆alkenyl, C₂₋₆alkynyl, optionally substituted phenyl, optionally substituted benzyl, optionally substituted benzoyl, optionally substituted 5- to 7-membered heteroaryl, wherein at least one of R, R₁, R₂, and R₃ comprises at least one radioactive isotope.

Certain preferred compounds of Formula II, include those compounds in which the R group comprises at least one radioactive isotope or more preferably one or more positron emitting radioactive isotopes. Yet other preferred compounds of Formula II include those compounds in which the R group comprises at least one radioactive isotope of carbon, fluorine, technetium, or iodine. Typically preferred radioactive isotopes of carbon, fluorine, technetium, and iodine, which are suitable for inclusion in the R group of compounds of Formula II include radioactive isotopes selected from ¹¹C, ¹⁸F, ⁹⁹Tc, ¹²³I, ¹²⁵I, and ¹³¹I.

Certain other preferred compounds of Formula II include those compounds in which R is C₁₋₆alkylthiol comprising at least one ¹¹C radionucleotide or R is NR₅R₆, R₅ is C₁₋₆alkyl comprising at least one ¹¹C radionucleotide; and R₆ is C₁₋₆alkyl, phenyl, benzyl, or benzoyl. Still other preferred compounds of Formula II include those compounds in which R is phenyl, furyl, thienyl, pyridinyl, pyrazinyl, pyrimidinyl, each of which is substituted with one or more substituents having at least one radioactive isotope selected from ¹¹C, ¹⁸F, ⁹⁹Tc, ¹²³I, ¹²⁵I, ¹³¹I or any combination thereof.

In yet other aspects, the invention provides compounds of Formula II, wherein the compounds have a structure according to Formula III: 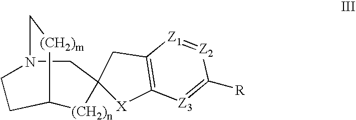
wherein m, n, R, Z₁, Z₂, and Z₃ are as defined in Formula II.

In still other aspects, the invention provides compounds of Formula II, wherein the compounds have a structure according to Formula IV: 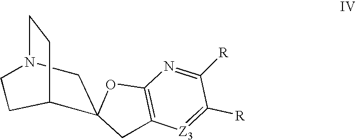
wherein

Z₃ is N or CH;
R is halogen, C₁₋₂fluoroalkyl, C₁₋₄alkoxy, C₁₋₂fluoroalkoxy, C₁₋₄alkylthio, formyl, carboxylate, N—C₁₋₄alkyl-N-benzylamino, N—C₁₋₄alkyl-N-benzoylamino, mono- and di-C₁₋₄alkylamino, or
R is phenyl or 5- or 6-membered heteroaryl substituted with one or more substituents selected from halogen, hydroxy, amino, cyano, formyl, C₁₋₄alkyl, C₂₋₄alkenyl, C₂₋₄alkynyl, C₁₋₄alkoxy, C₁₋₄alkylthio, C₁₋₄haloalkyl, C₁₋₄haloalkoxy.

Yet other preferred compounds of Formula IV include compounds having a structure according to Formula IVa 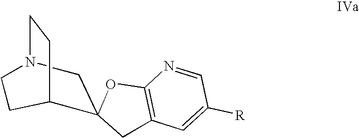
wherein
R is N—C₁₋₄alkyl-N-benzylamino, N—C₁₋₄alkyl-N-benzoylamino, mono- and di-C₁₋₄alkylamino, or
R is phenyl, furyl, thienyl, pyridinyl, pyrimidinyl, pyrazinyl, imidazolyl, or oxazolyl, each of which is substituted with one or more substituents selected from halogen, hydroxy, amino, cyano, formyl, C₁₋₄alkyl, C₂₋₄alkenyl, C₂₋₄alkynyl, C₁₋₄alkoxy, C₁₋₄alkylthio, C₁₋₄haloalkyl, C₁₋₄haloalkoxy.

Certain preferred compounds of Formula III, IV, or IVa, include those compounds in which the R group comprises at least one radioactive isotope or more preferably one or more positron emitting radioactive isotopes. Yet other preferred compounds of Formula III, IV, or IVa include those compounds in which the R group comprises at least one radioactive isotope of carbon, fluorine, technetium, or iodine. Typically preferred radioactive isotopes of carbon, fluorine, technetium, and iodine, which are suitable for inclusion in the R group of compounds of Formula III, IV, or IVa include radioactive isotopes selected from ¹¹C, ¹⁸F, ⁹⁹Tc, ¹²³I, ¹²⁵I, and ¹³¹I.

Certain other preferred compounds of Formula III, IV, or IVa include those compounds in which R is C₁₋₆alkylthiol comprising at least one ¹¹C radionucleotide or R is NR₅R₆, R₅ is C₁₋₆alkyl comprising at least one ¹¹C radionucleotide; and R₆ is C₁₋₆alkyl, phenyl, benzyl, or benzoyl. Still other preferred compounds of Formula II include those compounds in which R is phenyl, furyl, thienyl, pyridinyl, pyrazinyl, pyrimidinyl, each of which is substituted with one or more substituents having at least one radioactive isotope selected from ¹¹C, ¹⁸F, ⁹⁹Tc, ¹²³I, ¹²⁵I, ¹³¹I or any combination thereof.

Certain exemplary compounds of any one of Formulae III, IV, or IVa include those compounds in which R is selected from NHC(O)(4-¹¹C-methylthio-phenyl), NHC(O)(2-¹⁸F-fluoro-phenyl), NHC(O)(4-¹⁸F-fluoro-phenyl), N(¹¹C-methyl)(C(O)phenyl). Certain other exemplary compounds of Formulae III, IV, or Iva include those in which R is selected from the group consisting of IC-methyl, optionally substituted C₁₋₆alkyl, optionally substituted C₇₋₁₂aralkyl, optionally substituted C₆₋₁₂aryl, each of which may be substituted with one or more ¹¹C-methyl groups, ¹⁸F, ⁹⁹Tc, ¹²³I, ¹²⁵I, ¹³¹I, or a combination thereof. Still other preferred compounds of Formulae III, IV, or IVa, include those compounds in which R is ¹¹C-methyl, C₁₋₆alkyl substituted with one or more ¹⁸F, or benzyl substituted with one or more ¹²³I, ¹²⁵I, or ¹³¹I.

Certain other preferred compounds of Formula III, IV, or IVa include those compounds in which R comprises one or more radioisotope suitable for use in radiation therapy.

In another aspect, the invention provides compounds of Formula I, which have a structure according to Formula V: 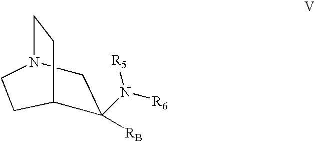
wherein
R₅ and R₆ are independently selected from hydrogen, C₁₋₆alkyl, C₂₋₆alkenyl, C₂₋₆alkynyl, optionally substituted phenyl, optionally substituted benzyl, optionally substituted benzoyl, optionally substituted 5- to 7-membered heteroaryl; and
R_{B} is hydrogen, C₁₋₆alkyl, C₂₋₆alkenyl, C₂₋₆alkynyl, or (C₃₋₈cycloalkyl)C₀₋₆alkyl.

Certain preferred compounds of Formula V have a structure according to Formula Va: 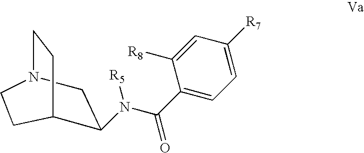
wherein
R₅ is hydrogen, methyl, or ¹¹C-methyl; and
R₇ and R₈ are independently selected at each occurrence from the group consisting of hydrogen, methyl, ¹¹C-methyl, ¹¹C-methoxy, ¹¹C-methylthiol, ¹⁸F, ¹²³I, and ¹²⁵I.

Certain preferred compounds of Formula V, include those compounds in which the R₅ or R₆ group comprises at least one radioactive isotope or more preferably one or more positron emitting radioactive isotopes. Yet other preferred compounds of Formula V include those compounds in which the R₅ or R₆ group comprises at least one radioactive isotope of carbon, fluorine, technetium, or iodine. Typically preferred radioactive isotopes of carbon, fluorine, technetium, and iodine, which are suitable for inclusion in the R₅ or R₆ group of compounds of Formula V include radioactive isotopes selected from ¹¹C, ¹⁸F, ⁹⁹Tc, ¹²³I, ¹²⁵I, and ¹³¹I.

Certain other preferred compounds of Formula Va include those compounds in which R₇ or R₈ is C₁₋₆alkylthiol comprising at least one ¹¹C radionucleotide, or at least one of R₇ and R₈ is ¹⁸F, ¹²³I, ¹²⁵I, ¹³¹I, or C₁₋₆alkylthiol comprising at least one ¹¹C radionucleotide, or more preferably at least one of R₇ or R₈ is ¹⁸F or ¹¹C-methylthiol. Still other preferred compounds of Formula V include those compounds in which R₇ or R₈ is phenyl, furyl, thienyl, pyridinyl, pyrazinyl, pyrimidinyl, each of which is substituted with one or more substituents having at least one radioactive isotope selected from ¹¹C, ¹⁸F, ⁹⁹Tc, ¹²³I, ¹²⁵I, ¹³¹I or any combination thereof.

Preferred compounds of the invention, particularly compounds suitable for use in the imaging methods provided by the invention, include one or more radioisotopes capable of emitting one or more forms of radiation which are suitable for detection with any standard radiology equipment such as PET, SPECT, gamma cameras, MRI and the like. Preferred radioisotopes include tritium and isotopes of carbon, fluorine, technetium, iodine and other isotopes capable of emitting positrons. Particularly preferred radioisotopes include ¹¹C, ¹⁸F, ⁹⁹Tc, ¹²³I, ¹²⁵I, and ¹³¹I.

Compounds of any one of Formula I, II, III, IV, IVa, V, or Va possess a binding affinity to α7 subtype nicotinic cholinergic receptors of 10 micromolar or less, more preferably of 1 micromolar or less, 100 nanomolar or less, 50 nanomolar or less, 25 nanomolar or less, 10 nanomolar or less, or most preferably of 1 nanomolar or less.

Particularly preferred compounds according to Formula I include the following non-limiting embodiments: (2′R)-N-¹¹C-methyl-N-(phenylmethyl)-spiro{1-azabicyclo[2.2.2]octane-3,2′(3′H)-furo[2,3-b]pyridin}-5′-amine, N-(R)-1-Aza-bicyclo[2.2.2]oct-3-yl-4-¹¹C-methylsulfanyl-benzamide, N-(R)-1-Aza-bicyclo[2.2.2]oct-3-yl-4-¹²⁵I-iodo-benzamide, (2′R)-5′-(2-¹²⁵I-Iodo-3-furanyl)spiro[1-azabicyclo[2.2.2]octane]-3,2′(3′H)-furo[2,3-b]pyridine, or (2′R)-5′-(2-¹⁸F-fluorophenyl)spiro[1-azabicyclo[2.2.2]octane]-3,2′(3′H)-furo[2,3-b]pyridine.

The present invention further provides method of imaging which comprise the steps of:
Providing at least one radiolabeled compound according to any one of Formula I, II, III, IV, IVa, V, or Va;
contacting cells or tissues with the radiolabeled compound; and
making a radiographic image.

The imaging methods of the invention are suitable for imaging any physiological process or feature in which α7 nicotinic cholinergic receptors are involved. Typically, imaging methods are suitable for identification of areas of tissues or targets which express high concentrations of α7 nicotinic cholinergic receptors. Preferred applications include imaging glutamateric neurotransmission, presynaptic glutamatergic neurotransmission, malignant tumors, lung cancer (including metastases), sensory gating, memory, or neuronal plasticity.

In certain aspects, the invention provides methods of imaging sensory gating, memory and/or neuronal plasticity with the imaging methods of the invention. The imaging methods of the invention which are suitable for use in imaging sensory gating, memory, or neuronal plasticity associated with Alzheimer's disease, schizophrenia, aging, head trauma, inflammation, nicotine addiction, or lung cancer.

In certain preferred imaging methods of the invention, the imaging agent according to any one of Formulae I, II, III, IV, IVa, V, or Va exhibits a binding selectivity ratio between α7-nicotinic cholinergic receptor and α4-nicotinic cholinergic receptors of at least about 5:1. More preferably, the binding selectivity ratio is at least 10:1, 50:1, or 100:1.

Preferred imaging methods provided by the invention include the use of I, II, III, IV, IVa, V, or Va which are capable of generating at least a 2:1 target to background ratio of radiation intensity, or more preferably about a 5:1, about a 10:1 or about a 15:1 ratio of radiation intensity between target and background.

In preferred methods of the invention the compounds of the invention are excreted from tissues of the body quickly to prevent prolonged exposure to the radiation of the radiolabeled compound administered to the patient. Typically compounds according to Formula I or any subformula thereof are eliminated from the body in less than about 24 hours. More preferably, compounds of the invention are eliminated from the body in less than about 16 hours, 12 hours, 8 hours, 6 hours, 4 hours, 2 hours, 90 minutes, or 60 minutes. Typically preferred compounds are eliminated in between about 60 minutes and about 120 minutes.

Preferred compounds of the invention are stable in vivo such that substantially all, e.g., more than about 50%, 60%, 70%, 80%, or more preferably 90% of the injected compound is not metabolized by the body prior to excretion.

Typical subjects to which compounds of the invention may be administered will be mammals, particularly primates, especially humans. For veterinary applications, a wide variety of subjects will be suitable, e.g. livestock such as cattle, sheep, goats, cows, swine and the like; poultry such as chickens, ducks, geese, turkeys, and the like; and domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids and cell samples of the above subjects will be suitable for use such as mammalian, particularly primate such as human, blood, urine or tissue samples, or blood urine or tissue samples of the animals mentioned for veterinary applications.

The present invention also provide packaged pharmaceutical compositions comprising a pharmaceutical acceptable carrier and a compound or salt of any one of Formula I, II, III, IV, IVa, V, or Va. In certain embodiments the packaged pharmaceutical composition will comprise the reaction precursors necessary generate the compound or salt according to Formula I or subformula thereof upon combination with a radiolabeled precursor. Other packaged pharmaceutical compositions provided by the present invention further comprise indicia comprising at least one of: instructions for using the composition to image cells or tissues expressing α7 nicotinic receptor, or instructions for using the composition to image sensory gating, memory, or neuronal plasticity in a patient suffering from Alzheimer's disease, schizophrenia, aging, head trauma, inflammation, nicotine addiction, or instructions for using the composition to image lung cancer.

In certain preferred embodiments, the invention provides a kit according to the invention contains from about 1 to about 30 mCi of the radionuclide-labeled imaging agent described above, in combination with a pharmaceutically acceptable carrier. The imaging agent and carrier may be provided in solution or in lyophilized form. When the imaging agent and carrier of the kit are in lyophilized form, the kit may optionally contain a sterile and physiologically acceptable reconstitution medium such as water, saline, buffered saline, and the like.

In another embodiment, the kit of the invention may contain the targeting molecule which has been covalently or non-covalently combined with a chelating agent; an auxiliary molecule such as mannitol, gluconate, glucoheptonate, tartrate, and the like; and a reducing agent such as SnCl₂, Na dithionite or tin tartrate. The targeting molecule/chelating agent and the auxiliary molecule may be present as separate components of the kit or they may be combined into one kit component. The unlabeled targeting molecule/chelating agent, the auxiliary molecule, and the reducing agent may be provided in solution or in lyophilized form, and these components of the kit of the invention may optionally contain stabilizers such as NaCl, silicate, phosphate buffers, ascorbic acid, gentisic acid, and the like. Additional stabilization of kit components may be provided in this embodiment, for example, by providing the reducing agent in an oxidation-resistant form.

Determination and optimization of such stabilizers and stabilization methods are well within the level of skill in the art. When the targeting molecule/chelating agent of this embodiment are in lyophilized form, the kit may optionally contain a sterile and physiologically acceptable reconstitution medium such as water, saline, buffered saline, and the like. The amounts of unlabeled targeting molecule/chelating agent, auxiliary molecule, and reducing agent in this embodiment are optimized in accordance with the methods for making the cardiovascular imaging agent set forth above. Radionuclides, including, but not limited to, ^{99m}Tc obtained from a commercially available ⁹⁹Mo/^{99m}Tc generator or commercially available ¹²³I, may be combined with the unlabeled targeting molecule/chelating agent and the reducing agent for a time and at a temperature sufficient to chelate the radionuclide to the targeting molecule/chelating agent, and the imaging agent thus formed is injected into the patient.

Imaging agents of the invention may be used in accordance with the methods of the invention by one of skill in the art, e.g., by specialists in nuclear medicine, to image sites having a high density of α7 nicotinic receptor concentration in a subject or patient. Any site of increased enzyme concentration may be imaged by the imaging methods and imaging agents of the present invention.

Images can be generated by virtue of differences in the spatial distribution of the imaging agents which accumulate at a site having a high density of α7 nicotinic receptor. The spatial distribution may be measured using any means suitable for the particular label, for example, a gamma camera, a PET apparatus, a SPECT apparatus, and the like. The extent of accumulation of the imaging agent may be quantified using known methods for quantifying radioactive emissions. A particularly useful imaging approach employs more than one imaging agent to perform simultaneous studies.

Preferably, a detectably effective amount of the imaging agent of the invention is administered to a subject. In accordance with the invention, “a detectably effective amount” of the imaging agent of the invention is defined as an amount sufficient to yield an acceptable image using equipment which is available for clinical use. A detectably effective amount of the imaging agent of the invention may be administered in more than one injection. The detectably effective amount of the imaging agent of the invention can vary according to factors such as the degree of susceptibility of the individual, the age, sex, and weight of the individual, idiosyncratic responses of the individual, the dosimetry. Detectably effective amounts of the imaging agent of the invention can also vary according to instrument and film-related factors. Optimization of such factors is well within the level of skill in the art.

The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

Chemical Description and Terminology

The compounds herein described may have one or more asymmetric centers or planes. Compounds of the present invention containing an asymmetrically substituted atom may be isolated in optically active or racemic forms. It is well known in the art how to prepare optically active forms, such as by resolution of racemic forms (racemates), by asymmetric synthesis, or by synthesis from optically active starting materials. Resolution of the racemates can be accomplished, for example, by conventional methods such as crystallization in the presence of a resolving agent, or chromatography, using, for example a chiral HPLC column. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral (enantiomeric and diastereomeric), and racemic forms, as well as all geometric isomeric forms of a structure are intended, unless the specific stereochemistry or isomeric form is specifically indicated.

When any variable occurs more than one time in any constituent or formula for a compound, its definition at each occurrence is independent of its definition at every other occurrence. Thus, for example, if a group is shown to be substituted with 0-2 R*, then said group may optionally be substituted with up to two R* groups and R* at each occurrence is selected independently from the definition of R*. Also, combinations of substituents and/or variables are permissible only if such combinations result in stable compounds.

As indicated above, various substituents of the various formulae (compounds of Formula I, II, III, IV, IVa, V, or Va) are “optionally substituted”, including R_{A}, R_{B}, R, R₁, R₂, R₃, R₄, R₅, R₆, R₇, R₈, Z₁, Z₂, or Z₃ of Formula I and subformulae thereof, and such substituents as recited in the sub-formulae such as Formula I and subformulae. The term “substituted,” as used herein, means that any one or more hydrogens on the designated atom or group is replaced with a selection from the indicated group of substituents, provided that the designated atom's normal valence is not exceeded, and that the substitution results in a stable compound. When a substituent is oxo (keto, i.e., ═O), then 2 hydrogens on an atom are replaced. The present invention is intended to include all isotopes (including radioisotopes) of atoms occurring in the present compounds.

When substituents such as R_{A}, R_{B}, R, R₁, R₂, R₃, R₄, R₅, R₆, R₇, R₈, Z₁, Z₂, or Z₃ of Formula I and subformulae thereof, and such substituents as recited in the sub-formulae are further substituted, they may be so substituted at one or more available positions, typically 1 to 3 or 4 positions, by one or more suitable groups such as those disclosed herein. Suitable groups that may be present on a “substituted” R₁, R₂, R₃ or other group include e.g., halogen; cyano; hydroxyl; nitro; azido; alkanoyl (such as a C₁₋₆ alkanoyl group such as acyl or the like); carboxamido; alkyl groups (including cycloalkyl groups, having 1 to about 8 carbon atoms, preferably 1, 2, 3, 4, 5, or 6 carbon atoms); alkenyl and alkynyl groups (including groups having one or more unsaturated linkages and from 2 to about 8, preferably 2, 3, 4, 5 or 6, carbon atoms); alkoxy groups having one or more oxygen linkages and from 1 to about 8, preferably 1, 2, 3, 4, 5 or 6 carbon atoms; aryloxy such as phenoxy; alkylthio groups including those having one or more thioether linkages and from 1 to about 8 carbon atoms, preferably 1, 2, 3, 4, 5 or 6 carbon atoms; alkylsulfinyl groups including those having one or more sulfinyl linkages and from 1 to about 8 carbon atoms, preferably 1, 2, 3, 4, 5, or 6 carbon atoms; alkylsulfonyl groups including those having one or more sulfonyl linkages and from 1 to about 8 carbon atoms, preferably 1, 2, 3, 4, 5, or 6 carbon atoms; aminoalkyl groups including groups having one or more N atoms and from 1 to about 8, preferably 1, 2, 3, 4, 5 or 6, carbon atoms; carbocyclic aryl having 6 or more carbons and one or more rings, (e.g., phenyl, biphenyl, naphthyl, or the like, each ring either substituted or unsubstituted aromatic); arylalkyl having 1 to 3 separate or fused rings and from 6 to about 18 ring carbon atoms, with benzyl being a preferred arylalkyl group; arylalkoxy having 1 to 3 separate or fused rings and from 6 to about 18 ring carbon atoms, with O-benzyl being a preferred arylalkoxy group; or a saturated, unsaturated, or aromatic heterocyclic group having 1 to 3 separate or fused rings with 3 to about 8 members per ring and one or more N, O or S atoms, e.g. coumarinyl, quinolinyl, isoquinolinyl, quinazolinyl, pyridyl, pyrazinyl, pyrimidyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl. Such heterocyclic groups may be further substituted, e.g. with hydroxy, alkyl, alkoxy, halogen and amino.

As used herein, “alkyl” is intended to include both branched and straight-chain saturated aliphatic hydrocarbon groups, having the specified number of carbon atoms. Examples of alkyl include, but are not limited to, methyl, ethyl, n-propyl, i-propyl, n-butyl, s-butyl, t-butyl, n-pentyl, and s-pentyl. Preferred alkyl groups are C₁₋₆ alkyl groups. Especially preferred alkyl groups are methyl, ethyl, propyl, butyl, and 3-pentyl. The term C₁₋₄ alkyl as used herein includes alkyl groups consisting of 1 to 4 carbon atoms, which may contain a cyclopropyl moiety. Suitable examples are methyl, ethyl, and cyclopropylmethyl.

“Cycloalkyl” is intended to include saturated ring groups, having the specified number of carbon atoms, such as cyclopropyl, cyclobutyl, cyclopentyl, or cyclohexyl. Cycloalkyl groups typically will have 3 to about 8 ring members.

In the term “(C₃₋₈ cycloalkyl)C₁₋₄ alkyl”, cycloalkyl, and alkyl are as defined above, and the point of attachment is on the alkyl group. This term encompasses, but is not limited to, cyclopropylmethyl, cyclohexylmethyl, and cyclohexylmethyl.

“Alkenyl” is intended to include hydrocarbon chains of either a straight or branched configuration comprising one or more unsaturated carbon-carbon bonds, which may occur in any stable point along the chain, such as ethenyl and propenyl. Alkenyl groups typically will have 2 to about 8 carbon atoms, more typically 2 to about 6 carbon atoms.

“Alkynyl” is intended to include hydrocarbon chains of either a straight or branched configuration comprising one or more carbon-carbon triple bonds, which may occur in any stable point along the chain, such as ethynyl and propynyl. Alkynyl groups typically will have 2 to about 8 carbon atoms, more typically 2 to about 6 carbon atoms.

“Haloalkyl” is intended to include both branched and straight-chain saturated aliphatic hydrocarbon groups having the specified number of carbon atoms, substituted with 1 or more halogen atoms. Examples of haloalkyl include, but are not limited to, mono-, di-, or tri-fluoromethyl, mono-, di-, or tri-chloromethyl, mono-, di-, tri-, tetra-, or penta-fluoroethyl, and mono-, di-, tri-, tetra-, or penta-chloroethyl. Typical haloalkyl groups will have 1 to about 8 carbon atoms, more typically 1 to about 6 carbon atoms.

“Alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Alkoxy groups typically have 1 to about 8 carbon atoms, more typically 1 to about 6 carbon atoms.

“Halolkoxy” represents a haloalkyl group as defined above with the indicated number of carbon atoms attached through an oxygen bridge.

As used herein, the term “alkylthio” includes those groups having one or more thioether linkages and preferably from 1 to about 8 carbon atoms, more typically 1 to about 6 carbon atoms.

As used herein, the term “alkylsulfinyl” includes those groups having one or more sulfoxide (SO) linkage groups and typically from 1 to about 8 carbon atoms, more typically 1 to about 6 carbon atoms.

As used herein, the term “alkylsulfonyl” includes those groups having one or more sulfonyl (SO₂) linkage groups and typically from 1 to about 8 carbon atoms, more typically 1 to about 6 carbon atoms.

As used herein, the term “alkylamino” includes those groups having one or more primary, secondary and/or tertiary amine groups and typically from 1 to about 8 carbon atoms, more typically 1 to about 6 carbon atoms.

“Halo” or “halogen” as used herein refers to fluoro, chloro, bromo, or iodo; and “counter-ion” is used to represent a small, negatively charged species such as chloride, bromide, hydroxide, acetate, sulfate, and the like.

As used herein, “carbocyclic group” is intended to mean any stable 3- to 7-membered monocyclic or bicyclic or 7- to 13-membered bicyclic or tricyclic group, any of which may be saturated, partially unsaturated, or aromatic. In addition to those exemplified elsewhere herein, examples of such carbocycles include, but are not limited to, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, phenyl, naphthyl, indanyl, and tetrahydronaphthyl.

As used herein, the term “heterocyclic group” is intended to include saturated, partially unsaturated, or unsaturated (aromatic) groups having 1 to 3 (preferably fused) rings with 3 to about 8 members per ring at least one ring containing an atom selected from N, O or S. The nitrogen and sulfur heteroatoms may optionally be oxidized. The term or “heterocycloalkyl” is used to refer to saturated heterocyclic groups.

The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. As used herein, the term “aromatic heterocyclic system” is intended to include any stable 5- to 7-membered monocyclic or 10- to 14-membered bicyclic heterocyclic aromatic ring system which comprises carbon atoms and from 1 to 4 heteroatoms independently selected from the group consisting of N, O and S. It is preferred that the total number of S and O atoms in the aromatic heterocycle is not more than 2, more preferably not more than 1.

Examples of heterocycles include, but are not limited to, those exemplified elsewhere herein and further include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofiranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

Preferred heterocyclic groups include, but are not limited to, pyridinyl, pyrimidinyl, furanyl, thienyl, pyrrolyl, pyrazolyl, pyrrolidinyl, morpholinyl, piperidinyl, piperazinyl, and imidazolyl. Also included are fused ring and spiro compounds containing, for example, the above heterocycles.

As used herein, the term “carbocyclic aryl” includes groups that contain 1 to 3 separate or fused rings and from 6 to about 18 ring atoms, without hetero atoms as ring members. Specifically preferred carbocyclic aryl groups include phenyl, and naphthyl including 1-napthyl and 2-naphthyl.

A “pharmaceutically acceptable carrier” refers to a biocompatible solution, having due regard to sterility, pH, isotonicity, stability, and the like and can include any and all solvents, diluents (including sterile saline, Sodium Chloride Injection, Ringer's Injection, Dextrose Injection, Dextrose and Sodium Chloride Injection, Lactated Ringer's Injection and other aqueous buffer solutions), dispersion media, coatings, antibacterial and antifungal agents, isotonic agents, and the like. The pharmaceutically acceptable carrier may also contain stabilizers, preservatives, antioxidants, or other additives, which are well known to one of skill in the art, or other vehicle as known in the art.

As used herein, “pharmaceutically acceptable salts” refer to derivatives of the disclosed compounds wherein the parent compound is modified by making non-toxic acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, malefic, hydroxymaleic, phenylacetic, glutamic, benzoic, salicylic, mesylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. The pharmaceutically acceptable salts of the present invention can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts may be found, e.g., in Remington's Pharmaceutical Sciences, 17th ed., Mack Publishing Company, Easton, Pa., p. 1418 (1985).

### EXAMPLES

The present invention is further illustrated by the following examples which should not be construed as limiting in any way. The contents of all cited references (including literature references, issued patents, published patent applications) as cited throughout this application are hereby expressly incorporated by reference. The practice of the present invention will employ, unless otherwise indicated, conventional techniques, which are within the skill of the art. Such techniques are explained fully in the literature.

General Chemistry

N,N-Dimethylformamide was distilled under reduced pressure from BaO. ¹H-NMR spectra were obtained with a Varian 400 (400 MHz) instrument. Chemical shifts are reported in ppm (¹H) relative to internal tetramethylsilane in CDCl₃. High resolution mass spectrometry was performed at the University of Minnesota Mass Spectrometry facility. Elemental Analyses were determined by Quantitative Technologies Inc. (Whitehouse, N.J.). Short-path column chromatography was performed using E. Merck 7729 (<230 mesh) silica gel. [¹¹C]iodomethane was prepared with the GE PETtrace MeI MicroLab (Milwaukee, Wis.) using a PETtrace biomedical cyclotron. HPLC equipment consisted of Rheodyne 7126 injectors, Waters 590 EF pumps, Waters 440 UV absorbance detector (254 nm), and a NaI(TI) crystal (2 inch) scintillation detector. Hewlett-Packard 3390A integrators and a Rainin Dynamax system were used to record and analyze high performance liquid chormatography (HPLC) chromatograms. Semi-preparative (10×250 mm) and analytical (4.6×250 mm) reversed phase HPLC columns (Phenomenex Luna C-18, 10 mm) were used, respectively, for purification and quality control of the radiotracers.

Radiochemical Syntheses

After reaction with [¹¹C]CH₂O, generated in situ from [¹¹C]-CO₂, compound 1 was produced in 6.5% radiochemical yield at 26 min after end-of-synthesis (e.o.s) (Scheme 1). A specific radioactivity of 61 GBq/μmol (1,649 Ci/mmol) was obtained.

The disulfide bond of the precursor to 2 was reduced and the resulting thiol reacted with [¹¹C]iodomethane to give the radiomethylated adduct in 33% yield with specific radioactivities that ranged from 152-216 GBq/μmol (4,095-5,828 Ci/mmol) with an average (n=2) of 184 GBq/μmol (4,962 Ci/mmol) calculated at the e.o.s. (Scheme 1).

Compound 3 was synthesized from the quinuclidyl bromobenzamide precursor by a copper-assisted iodo-debromination in 57% yield at a specific radioactivity of 74 GBq/μmol (1,990 Ci/mmol) (Scheme 2).

Compound 4 was made by treatment with [¹²⁵I]NaI according to the chloramine-T method in 13% yield at a specific activity of >74 GBq/μmol (>2,000 Ci/mmol) (Scheme 2). An unlabeled analog of 4 was synthesized and characterized to provide a standard for HPLC.

Compounds 1-4 were prepared by the procedures illustrated in Schemes 1 and 2 and further discussed in Examples 1-5.

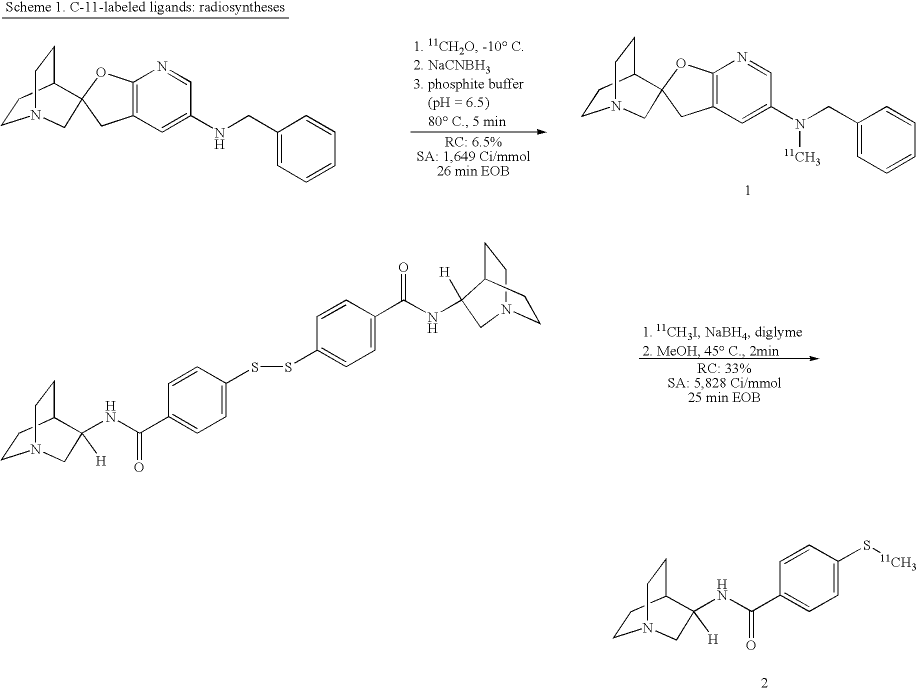

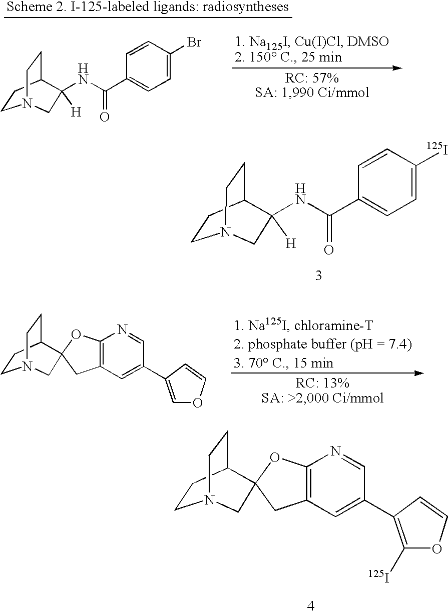

### Example 1

(2′R)-N-[¹¹C]methyl-N-(phenylmethyl)-spiro[1-azabicyclo[2.2.2]octane-3,2′(3′H)-furo[2,3-b]pyridin]-5′-amine 1 (Scheme 1). [1′C]Formaldehyde was generated in situ to synthesize 1 (25). [¹¹C]CO₂ was bubbled into a solution of lithium aluminum hydride in THF (1.0M, 0.6 mL) at −10° C. in a 15% sodium chloride/ice bath, followed by the addition of 2M H₂SO₄ (0.6 mL). The ice bath was removed and [¹¹C]CH₂O was bubbled into a v-vial containing the desmethyl precursor (2.0 mg) in phosphite buffer pH 6.5 (0.4 mL) (26). The reaction was heated at 80° C. for 10 min before quenching with 500 FL of HPLC mobile phase consisting of 30:60 acetonitrile/water in 0.1 M ammonium formate. The crude reaction was purified by reversed phase HPLC using the above mobile phase at 8 mL/min. The radioactive product (t_{R}=9.0 min), which was resolved from the precursor (t_{R}=5.3 min), was collected remotely. After concentration to dryness under reduced pressure and heat (80° C.), the radiotracer was reconstituted in sterile 0.9% saline (7.0 mL), and passed through a 0.2 μm sterile filter (Acrodisc, Gelman) into a sterile, pyrogen-free multi-dose vial. Sterile NaHCO₃ (3.0 mL, 8.4%) was added to give a final formulation of pH 7.4. Specific radioactivity was calculated by relating radioactivity to the mass associated with the UV absorbance peak of carrier.

### Example 2

N—(R)-1-aza-bicyclo[2.2.2]oct-3-yl-4-[¹¹C]methylsulfanyl-benzamide 2 (Scheme 1). [¹¹C]Iodomethane, carried by a stream of nitrogen, was trapped in a dry ice/ethanol cooled solution of the disulfide precursor (0.5-1.0 mg) in anhydrous MeOH (0.1 mL) and sodium borohydride in tetraglyme (3M, 0.1 mL). The reaction was heated at 45° C. for 2 min before quenching with 200 μL of HPLC mobile phase consisting of 0.1% TFA in 20:80 acetonitrile/water. The crude reaction was purified by reversed phase HPLC using the above mobile phase at 12 mL/min. The radioactive product (t_{R}=8.9 min), which was well separated from the precursor (t_{R}=5.5 min), was remotely collected. A single radioactive peak (t_{R}=2.3 min) corresponding to authentic 2 was observed. The radiotracer was formulated in the same manner as for 1. Specific radioactivities were calculated in a manner identical to that for 1.

### Example 3

N-(R)-1-Aza-bicyclo[2.2.2]oct-3-yl-4-[¹²⁵I]iodo-benzamide 3 (Scheme 2). The free base of the brominated precursor to 3 was prepared by dissolving 1 mg in 0.4 mL of water, adding 0.1 mL of 0.1 M NaOH, extracting with dichloromethane (0.3 mL) and evaporating the organic phase to dryness. The residue was taken up by methylsulfoxide (10 μL) and transferred to a 1 mL v-vial. To the vial was added 5 μL of [¹²⁵I]NaI, (11.7 MBq, 314 μCi) and 20 μL of a methylsulfoxide solution of Cu(I)Cl (10 mg/mL) (27), (20). The sealed vial was placed in a 150° C. sand bath and heated for 25 min. The reaction was cooled and subsequently quenched by addition of 0.4 mL mobile phase (25/75 acetontrile/water in 0.1 M ammonium formate). The crude reaction mixture was purified on a C-18 Luna column 10×250 mm at a flow rate of 6 mL/min. The desired radioactive peak (t_{R}=30 min) was well-resolved from bromo precursor (t_{R}=19 min) and the radiotracer was collected. The radioligand was concentrated by rotary evaporation at 45° C. and formulated in saline [ca. 74 KBq (2 μCi) per 0.2 mL] for mouse biodistribution studies. A semi-preparative C-18 Luna column using a mobile phase of 25/75 acetontrile/water in 0.1 M ammonium formate at 12 mL/min was employed to estimate specific radioactivity and confirm radiochemical purity.

### Example 4

(2′R)-5′-(2-[¹²⁵I]iodo-3-furanyl)spiro[1-azabicyclo[2.2.2]octane]-3,2′(3′H)-furo[2,3-b]pyridine 4 (Scheme 2). To a 0.5 mL v-vial containing 25 μL of the spirofuropyridine precursor (0.35 mg in 0.1 mL phosphate buffer) was added [¹²⁵I]NaI (81 μBq, 2.2 mCi) and 25 μL of an aqueous solution of chloramine-T (2.3 mg/mL). The sealed vial was placed in a 70° C. sand bath and heated for 15 min. The reaction was cooled and subsequently quenched by addition of 50 μL of sodium metabisulfite (0.1μ) and 100 μL of mobile phase (30/70 acetontrile/water in 0.1 M ammonium formate). The crude reaction mixture was purified on a C-18 Luna column 10×250 mm at a flow rate of 6 mL/min. The desired radioactive peak (t_{R}=8.9 min) was resolved from precursor (t_{R}=6.3 min) and the radiotracer was collected. The radioligand was concentrated by rotary evaporation at 45° C. and formulated in saline [ca. 74 KBq (2 μCi) per 0.2 mL] for mouse biodistribution studies. The semi-preparative C-18 luna column using a mobile phase of 30/70 acetontrile/water in 0.1 M ammonium formate at 6 mL/min was employed to estimate specific radioactivity and confirm radiochemical purity.

### Example 5

Synthesis of Unlabeled 4. A solution of the spirofuropyridine precursor (0.9 mg, 3.2 μmol) in dichloromethane (70 μL) was added to mercuric acetate (1.5 mg, 3.3 μmol) in dichloromethane (70 μL). The mixture was stirred for 15 min. A solution of 12 (1 mg, 3.2 μmol) in dichloromethane was added dropwise and stirred for 30 min. The reaction mixture was diluted with chloroform (3 mL), filtered, washed with 5% sodium thiosulfate (5 mL), water (5 mL), brine (5 mL), and dried. The residue was purified by reversed phase HPLC using mobile phase consisting of 35:65 acetonitrile/water in 0.1 M ammonium formate at 6 mL/min (1.2 mg, yield 91.9%).

¹H-NMR (CDCl₃, δ) 1.55-1.60 (m, 2H), 1.77-1.89 (m, 2H), 2.30 (t, J=0.8 Hz, 2H), 2.90 (t, J=7.2 Hz, 2H), 3.00-3.13 (m, 4H), 3.46 (t, J=10.0 Hz, 2H), 6.52 (d, J=2.0 Hz, 1H), 7.65 (d, J=2.0 Hz, 1H), 7.66 (m, 1H), 8.18 (m, 1H). HRMS-CI: m/z calcd. 409.0413; found 409.0439 (M+H)⁺.

### Example 6

The synthesis of (2′R)-5′-(2-¹⁸F-fluorophenyl)spiro[1-azabicyclo[2.2.2]octane]-3,2′(3′H)-furo[2,3-b]pyridine can be prepared by the processes depicted in Scheme 3, which have previously been used to prepare the ¹⁹F-fluorine analog.

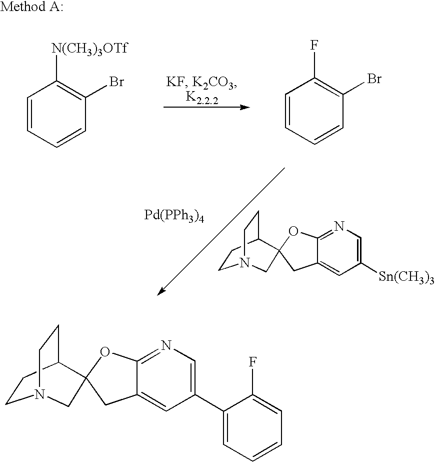

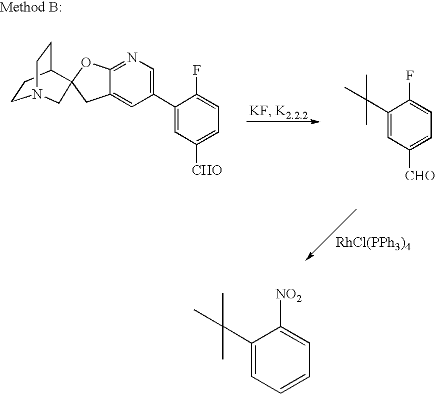

### Example 7

Receptor Binding Assays. The affinities of 1-4 for the rat α4β2 and α7 nAChRs were determined as described previously (Mullen, et al., J. Med. Chem.; 2000;43(22):4045-50). Affinities for the rat 5-HT₃ receptor were also determined as described previously (Macor, et al., Bioorg. Med. Chem. Lett. 2001;11(3):319-21). All Kᵢ determinations were done using 5-7 concentrations. The relative affinities (Kᵢ) of the nonradioactive analogs of 1-4 ranged from 0.26 to 16 nM (Table 1). There was a wide margin in selectivity compared to the α4β2 receptor subtype. Also included are the relative binding affinities to the 5-HT₃ receptor, which has high sequence homology to the α7-nAChR.

TABLE 1
Binding Affinities and Selectivities

| | label | Kᵢ, nM | α7/α4 | α7/5-HT₃ |
|---|---|---|---|---|
| 1 | C-11 | 0.54 (1) | >22,000 (1) | 18 (1) |
| 2 | C-11 | 5.8 (3) | 14,000 (1) | 1100 (1) |
| 3 | I-125 | 18 (3) | 36000 (1) | 280 (1) |
| (precursor)* | | | | |
| 4 | I-125 | 0.26 (2) | NP^{†} | NP^{†} |

Number of determinations in parenthesis
*values refer to the precursor to the radiolabeled compound
^{†}Not performed

### Example 8

Physical Properties of 1-4

The physical properties of 1-4 are depicted in Table 2. The Log D_{7.4} values and molecular weights indicate that those compounds should gain ready access to the brain. ACD log P was calculated using a log P prediction method as implimented in ACD UNIX Batch V4.5. It is also based on a fragment contribution method.

TABLE 2
Physical Properties of Potential Ligands

| Compound | MW | Log D_{7.4} | Ndonors | Lipinski Score |
|---|---|---|---|---|
| 1 | 335 | 1.25 | 0 | 0 |
| 2 | 276 | 1.32 | 1 | 0 |
| 3 | 356 | 1.78 | 1 | 0 |
| 4 | 408 | 2.00 | 0 | 0 |

### Example 9

Animal Studies

Rodent In Vivo Biodistribution Studies of 1-4. Male CD-1 mice weighing between 20 and 25 g received an injection of 3.7 MBq (100 μCi) for 1 and 2 and 0.67 MBq (2 μCi) for 3 and 4 through the tail vein. The corresponding amounts administered were 0.2-0.8 μg/kg for 1 and 2 and 0.01-0.02 μg/kg for 3 and 4. For kinetic studies of 1-4, the mice were killed by cervical dislocation at 5, 15, 30 and 60 min after intravenous injection of radiotracer in 200 μL saline vehicle. The brains were removed and placed on ice, and the cerebellum, olfactory bulb, hypothalamus, hippocampus, striatum, cortex, brain stem and thalamus were harvested. A 45 min time point was obtained for 2 and one at 120 min for 3 and 4. The tissue samples were weighed and their radioactivity determined in an automated □ counter (1282 Compugamma CS; Pharmacia/LKB Nuclear Inc., Gaithersburg, Md.). The radioactivity concentration in aliquots of the injected tracer were determined along with the samples and served as standards for the calculation of percentage injected dose per gram of tissue (% ID/g). To assess binding specificity, subgroups of mice were treated with 10 mg/kg nicotine (for D or increasing doses of unlabeled 4 (0.01-10 mg/kg) 5 min prior to injection of radiotracer. Mice were killed at 30 and 60 min after blockade for 3 and 4, respectively.

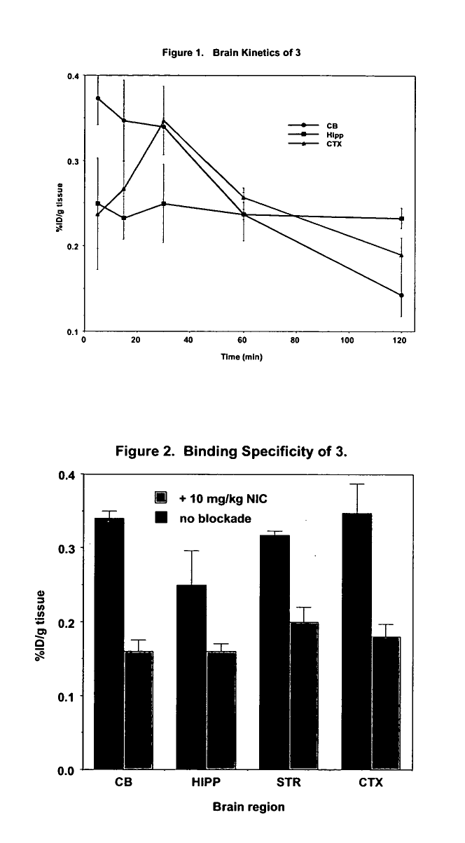

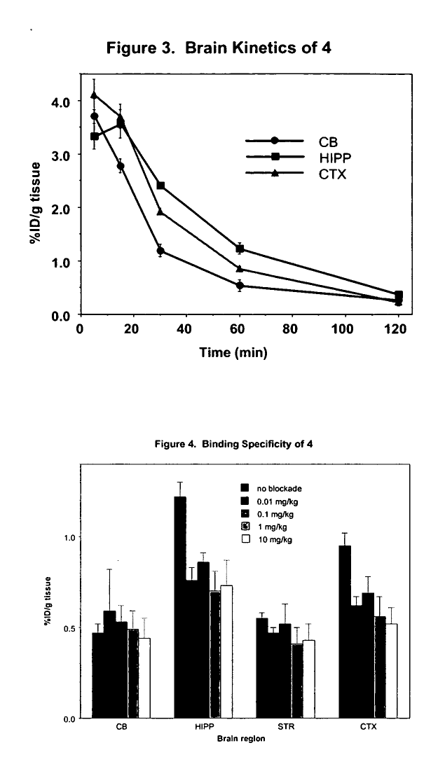

No α7-nAChR-selective, regional brain uptake at 5, 15, 30 and 60 minutes for compound 1 (Table 3). Also shown are uptake values for low specific activity (LSA) determinations at 30 and 60 min. No regional selectivity was demonstrated, nor was there evidence of receptor blockade in the LSA studies. As with the other radiotracers in this study (except for 2), compound 1 obtains limited but measurable uptake within the brain. We also observed limited uptake of 2 within the brain at all time points, with no regional selectivity (Table 4). Although compound 3 does not attain high uptake values within the brain, at values <0.4% ID/g, retention within hippocampus, a target tissue, was demonstrated (FIG. 1). That observation was sufficiently encouraging to warrant a blocking study that failed to demonstrate regionally-selective blockade, although significantly decreased radiotracer uptake was demonstrated in the regions studied (FIG. 2). Compound 4 gained ready access to the brain with modest but higher uptake in target tissue (hippocampus) than within other brain substructures (FIG. 3). The receptor blockade study indicates a significant blockade in the target tissue and slightly less so in cortex, which also contains α7-nAChRs (FIG. 4).

TABLE 3
Brain Distribution of Compound 1
% ID/g ± SD (n = 3)

| Tissue | 5 min | 15 min | 30 min | 60 min | 30 min LSA* | 60 min LSA |
|---|---|---|---|---|---|---|
| CB | 1.66 ± 0.50 | 1.38 ± 0.32 | 1.16 ± 0.19 | 0.84 ± 0.05 | 1.00 ± 0.06 | 0.65 ± 0.07 |
| HIPP | 1.24 ± 0.33 | 1.12 ± 0.15 | 0.91 ± 0.05 | 0.81 ± 0.10 | 1.27 ± 0.09 | 0.98 ± 0.16 |
| STR | 1.47 ± 0.38 | 1.20 ± 0.15 | 0.93 ± 0.10 | 0.69 ± 0.06 | 1.20 ± 0.05 | 0.75 ± 0.22 |
| F. CTX | 1.77 ± 0.36 | 1.38 ± 0.25 | 0.95 ± 0.06 | 0.79 ± 0.11 | 1.28 ± 0.07 | 1.07 ± 0.15 |
| CTX | 1.46 ± 0.48 | 1.28 ± 0.21 | 0.89 ± 0.11 | 0.69 ± 0.10 | 1.16 ± 0.13 | 0.86 ± 0.15 |
| THAL | 1.61 ± 0.61 | 1.35 ± 0.28 | 1.11 ± 0.06 | 0.77 ± 0.10 | 1.23 ± 0.12 | 0.81 ± 0.06 |
| S. COLL | 1.49 ± 0.50 | 1.42 ± 0.26 | 1.09 ± 0.14 | 0.88 ± 0.16 | 1.04 ± 0.15 | 0.88 ± 0.16 |

*LSA = low specific activity

TABLE 4
Brain Distribution of Compound 2
% ID/g (n = 4)

| Tissue | 5 min | 15 min | 30 min | 45 min | 60 min |
|---|---|---|---|---|---|
| CB | 0.23 ± 0.01 | 0.26 ± 0.04 | 0.14 ± 0.03 | 0.09 ± 0.01 | 0.08 ± 0.01 |
| HIPP | 0.15 ± 0.01 | 0.24 ± 0.04 | 0.14 ± 0.03 | 0.13 ± 0.02 | 0.12 ± 0.02 |
| STR | 0.22 ± 0.06 | 0.37 ± 0.05 | 0.16 ± 0.03 | 0.15 ± 0.05 | 0.18 ± 0.05 |
| CTX | 0.16 ± 0.04 | 0.17 ± 0.03 | 0.14 ± 0.05 | 0.10 ± 0.02 | 0.10 ± 0.02 |
| THAL | 0.18 ± 0.02 | 0.26 ± 0.09 | 0.13 ± 0.03 | 0.10 ± 0.01 | 0.14 ± 0.03 |
| HYPO | 0.30 ± 0.02 | 0.55 ± 0.05 | 0.22 ± 0.04 | 0.22 ± 0.09 | 0.27 ± 0.05 |

The disclosures of all articles and references mentioned in this application, including patents, are incorporated herein by reference.

The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

1. A compound, or pharmaceutically acceptable salt thereof, according to Formula I: 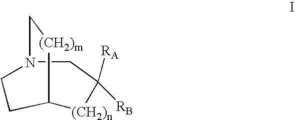
wherein
m and n are independently selected from 0 or 1;
R_{A} is NR₅R₆;
R_{B} is hydrogen, C₁₋₆alkyl, C₂₋₆alkenyl, C₂₋₆alkynyl, or (C₃₋₈cycloalkyl)C₀₋₆alkyl; or
R_{A} and R_{B}, taken in combination, form a substituted heterocycle or a substituted benzoheterocycle; and
R₅ and R₆ are independently selected from hydrogen, C₁₋₆alkyl, C₂₋₆alkenyl, C₂₋₆alkynyl, optionally substituted phenyl, optionally substituted benzyl, optionally substituted benzoyl, optionally substituted 5- to 7-membered heteroaryl, wherein the compound of Formula I comprises at least one radioisotope.

2. A compound of claim 1, wherein the compound has a structure according to Formula II 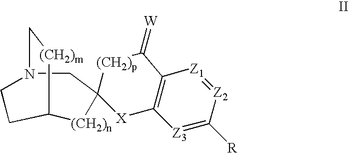
wherein
m, n, and p are independently selected from 0 or 1;
W is O, F₂ or H₂;
X is O or S;
Z₁ is N or CR₁;
Z₂ is N or CR₂;
Z₃ is N or CR₃, wherein one or two of Z₁, Z₂, and Z₃ is nitrogen;
R is halogen, C₁₋₆haloalkyl, C₁₋₆alkoxy, C₁₋₆haloalkoxy, C₁₋₆alkylthio, formyl, carboxylate, —NR₅R₆, substituted aryl, or substituted heteroaryl;
R₁, R₂, and R₃ are independently selected at each occurrence from the group consisting of hydrogen, halogen hydroxy, amino, cyano, nitro, C₁₋₆alkyl, C₂₋₆alkenyl, C₂₋₆alkynyl, C₁₋₆alkoxy, mono- or di-C₁₋₆alkylamino, optionally substituted aryl or optionally substituted heteroaryl; and
R₅ and R₆ are independently selected from hydrogen, C₁₋₆alkyl, C₂₋₆alkenyl, C₂₋₆alkynyl, optionally substituted phenyl, optionally substituted benzyl, optionally substituted benzoyl, optionally substituted 5- to 7-membered heteroaryl, wherein at least one of R, R₁, R₂, and R₃ comprises at least one radioactive isotope.

3. The compound of claim 2, wherein the R group comprises at least one radioactive isotope.

4. The compound of claim 2, wherein the R group comprises at least one radioactive isotope of carbon, fluorine, technetium, or iodine.

5. The compound of claim 2, wherein the R group comprises one or more positron emitting radioactive isotopes.

6. The compound of claim 2, wherein the R group comprises one or more radioactive isotope selected from ¹¹C, ¹⁸F, ⁹⁹Tc, ¹²³I, ¹²⁵I, ¹³¹I or any combination thereof.

7. The compound of claim 2, wherein R is C₁₋₆alkylthiol comprising at least one ¹¹C. radionucleotide.

8. The compound of claim 2, wherein R is NR₅R₆, R₅ is C₁₋₆alkyl comprising at least one ¹¹C radionucleotide; and R₆ is C₁₋₆alkyl, phenyl, benzyl, or benzoyl.

9. The compound of claim 2, wherein R is phenyl, furyl, thienyl, pyridinyl, pyrazinyl, pyrimidinyl, each of which is substituted with one or more substituents having at least one radioactive isotope selected from ¹¹C, ¹⁸F, ⁹⁹Tc, ¹²³I, ¹²⁵I, ¹³¹I or any combination thereof.

10. The compound of claim 2, wherein the compound has a structure according to Formula III:

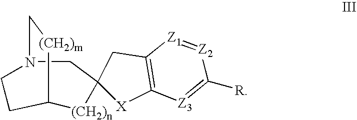

11. The compound of claim 10, wherein the compound has a structure according to Formula IV 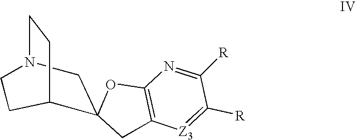
wherein
Z₃ is N or CH;
R is halogen, C₁₋₂fluoroalkyl, C₁₋₄alkoxy, C₁₋₂fluoroalkoxy, C₁₋₄alkylthio, formyl, carboxylate, N—C₁₋₄alkyl-N-benzylamino, N—C₁₋₄alkyl-N-benzoylamino, mono- and di-C₁₋₄alkylamino, or
R is phenyl or 5- or 6-membered heteroaryl substituted with one or more substituents selected from halogen, hydroxy, amino, cyano, formyl, C₁₋₄alkyl, C₂₋₄alkenyl, C₂₋₄alkynyl, C₁₋₄alkoxy, C₁₋₄alkylthio, C₁₋₄haloalkyl, C₁₋₄haloalkoxy.

12. The compound of claim 11, wherein the compound has a structure according to Formula IVa 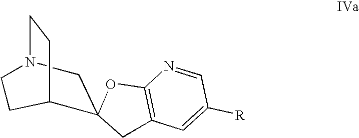
wherein
R is N—C₁₋₄alkyl-N-benzylamino, N—C₁₋₄alkyl-N-benzoylamino, mono- and di-C₁₋₄alkylamino, or
R is phenyl, furyl, thienyl, pyridinyl, pyrimidinyl, pyrazinyl, imidazolyl, or oxazolyl, each of which is substituted with one or more substituents selected from halogen, hydroxy, amino, cyano, formyl, C₁₋₄alkyl, C₂₋₄alkenyl, C₂₋₄alkynyl, C₁₋₄alkoxy, C₁₋₄alkylthio, C₁₋₄haloalkyl, C₁₋₄haloalkoxy.

13. The compound of claim 11, wherein the R group comprises at least one radioactive isotope.

14. The compound of claim 11, wherein R comprises at least one positron emitting isotope of carbon, fluorine, technetium, or iodine.

15. The compound of claim 11, wherein R comprises one or more radioisotopes selected from ¹¹C, ¹⁸F, ⁹⁹Tc, ¹²³I, ¹²⁵I, ¹³¹I or any combination thereof.

16. The compound of claim 12, wherein R is selected from NHC(O)(4-¹¹C-methylthio-phenyl), NHC(O)(2-¹⁸F-fluoro-phenyl), NHC(O)(4-¹⁸F-fluoro-phenyl), N(¹¹C-methyl)(C(O)phenyl), and 2-¹⁸F-fluoro-phenyl,

17. The compound of claim 12, wherein R is selected from the group consisting of ¹¹C-methyl, optionally substituted C₁₋₆alkyl, optionally substituted C₇₋₁₂aralkyl, optionally substituted C₆₋₁₂aryl, each of which may be substituted with one or more ¹¹C-methyl groups, ¹⁸F, ⁹⁹Tc, ¹²³I, ¹²⁵I, ¹³¹I, or a combination thereof.

18. The compound of claim 17, wherein R is ¹¹C-methyl, C₁₋₆alkyl substituted with one or more ¹⁸F, or benzyl substituted with one or more ¹²³I, ¹²⁵I, or ¹³¹I.

19. The compound of claim 11, wherein R comprises one or more radioisotope suitable for use in radiation therapy.

20. The compound of claim 1, wherein the compound is according to Formula V: 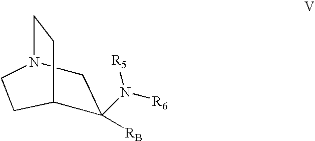
wherein
R₅ and R₆ are independently selected from hydrogen, C₁₋₆alkyl, C₂₋₆alkenyl, C₂₋₆alkynyl, optionally substituted phenyl, optionally substituted benzyl, optionally substituted benzoyl, optionally substituted 5- to 7-membered heteroaryl; and
R_{B} is hydrogen, C₁₋₆alkyl, C₂₋₆alkenyl, C₂₋₆alkynyl, or (C₃₋₈cycloalkyl)C₀₋₆alkyl.

21. The compound of claim 20, wherein the compound is according to Formula Va: 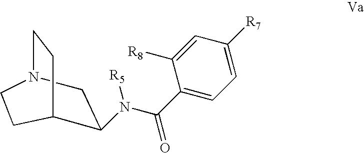
wherein
R₅ is hydrogen, methyl, or ¹¹C-methyl; and
R₇ and R₈ are independently selected at each occurrence from the group consisting of hydrogen, methyl, ¹¹C-methyl, ¹¹C-methoxy, ¹¹C-methylthiol, ¹⁸F, ¹²³I, and ¹²⁵I.

22. A compound of claim 1 which has a binding affinity to a7-nicotinic cholinergic receptor of less than about 10 μM.

23. A compound of claim 1 which has a binding affinity to a7-nicotinic cholinergic receptor of less than about 1 μM.

24. A compound of claim 1 which has a binding affinity to a7-nicotinic cholinergic receptor of less than about 100 nM.

25. A compound of claim 1 which has a binding affinity to a7-nicotinic cholinergic receptor of less than about 10 nM.

26. A compound of claim 1 which has a binding affinity to a7-nicotinic cholinergic receptor of less than about 1 nM.

27. A pharmaceutical composition comprising a pharmaceutically acceptable carrier and a compound or salt of claim 1.

28. A package comprising a pharmaceutical composition of claim 27 in a container and further comprising indicia comprising at least one of:
instructions for using the composition to image cells or tissues expressing a7-nicotinic cholinergic receptor, or
instructions for using the composition to image sensory gating, memory, or neuronal plasticity in a patient suffering from a neurological disease or disorder, or
instructions for using the composition to image lung cancer.

29. An imaging method comprising the steps of:
providing a radiolabeled compound according to Formula I: 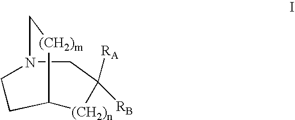
wherein
m and n are independently selected from 0 or 1;
R_{A} is NR₅R₆;
R_{B} is hydrogen, C₁₋₆alkyl, C₂₋₆alkenyl, C₂₋₆alkynyl, or (C₃₋₈cycloalkyl)C₀₋₆alkyl; or
R_{A} and R_{B}, taken in combination, form a substituted heterocycle or a substituted benzoheterocycle; and
R₅ and R₆ are independently selected from hydrogen, C₁₋₆alkyl, C₂₋₆alkenyl, C₂₋₆alkynyl, optionally substituted phenyl, optionally substituted benzyl, optionally substituted benzoyl, optionally substituted 5- to 7-membered heteroaryl, wherein the compound of Formula I comprises at least one radioisotope; or a pharmaceutically acceptable salt thereof; contacting cells or tissues with the radiolabeled compound; and making a radiographic image.

30. The method of claim 29, wherein the compound of Formula I is a compound having a structure according to Formula IVa: 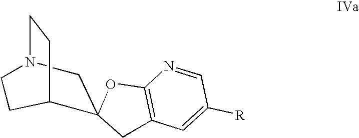
wherein
R is N—C₁₋₄alkyl-N-benzylamino, N—C₁₋₄alkyl-N-benzoylamino, mono- and di-C₁₋₄alkylamino, or
R is phenyl, furyl, thienyl, pyridinyl, pyrimidinyl, pyrazinyl, imidazolyl, or oxazolyl, each of which is substituted with one or more substituents selected from halogen, hydroxy, amino, cyano, formyl, C₁₋₄alkyl, C₂₋₄alkenyl, C₂₋₄alkynyl, C₁₋₄alkoxy, C₁₋₄alkylthio, C₁₋₄haloalkyl, C₁₋₄haloalkoxy.

31. The method of claim 29, wherein the compound of Formula I is a compound having a structure according to Formula Va: 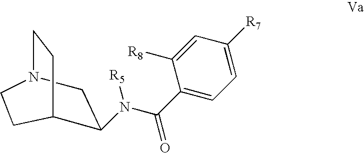
wherein
R₅ is hydrogen, methyl, or ¹¹C-methyl; and
R₇ is hydrogen, methyl, ¹¹C-methyl, ¹¹C-methoxy, ¹¹C-methylthiol, ¹⁸F, ¹²³I, or ¹²⁵I.

32. The method of claim 29, wherein the imaging method is suitable for use in imaging sensory gating, memory, or neuronal plasticity.

33. The method of claim 29, wherein the imaging method is suitable for use in imaging sensory gating, memory, or neuronal plasticity associated with Alzheimer's disease, schizophrenia, aging, head trauma, inflammation, nicotine addiction, or lung cancer.

34. The method of claim 29, wherein the imaging method is suitable for imaging of cancer which expresses a7-nicotinic cholinergic receptor.

35. The method of claim 29, wherein the imaging method is suitable for imaging of lung cancer.

36. The method of claim 29, wherein the imaging method is suitable for imaging lung cancer including metastases.

37. The method of claim 29, wherein the radiolabeled compound exhibits a selectivity for a7-nicotinic cholinergic receptor to a4-nicotinic cholinergic receptors of at least about 5:1.

38. The method of claim 29, wherein the radiolabeled compound is stable in vivo.

39. The method of claim 29, wherein the radiolabeled compound substantially localizes to a site or sites expressing a7-nicotinic cholinergic receptor, within about 120 minutes after administration.

40. The method of claim 29, wherein the radiolabeled compound substantially localizes to a site or sites expressing a7-nicotinic cholinergic receptor, within about 60 minutes after administration.

41. The method of claim 29, wherein the radiolabeled compound substantially localizes to a site or sites expressing a7-nicotinic cholinergic receptor, within about 30 minutes after administration.

42. The method of claim 29, wherein the radiolabeled compound is detected by a gamma camera. positron emission tomography (PET) or single photon emission tomography (SPECT).

43. The method of claim 29, wherein the subject is a human, rat, mouse, cat, dog, horse, sheep, cow, monkey, avian, or amphibian.

44. A compound selected from the group consisting of (2′R)-N-¹¹C-methyl-N-(phenylmethyl)-spiro{1-azabicyclo[2.2.2]octane-3,2′(3′H)-furo[2,3-b]pyridin}-5′-amine, N-(R)-1-Aza-bicyclo[2.2.2]oct-3-yl-4-¹¹C-methylsulfanyl-benzamide, N-(R)-1-Aza-bicyclo[2.2.2]oct-3-yl-4-¹²⁵I-iodo-benzamide, (2′R)-5′-(2-¹²⁵I-Iodo-3-furanyl)spiro[1-azabicyclo[2.2.2]octane]-3,2′(3′H)-furo[2,3-b]pyridine, or (2′R)-5′-(2-¹⁸F-fluorophenyl)spiro[1-azabicyclo[2.2.2]octane]-3,2′(3′H)-furo[2,3-b]pyridine.
